(19) United States
(12) Patent Application Publication
(10) Pub. No.: US 2018/0228412 A1
(43) Pub. Date: Aug. 16, 2018

(54) SPECTROSCOPY THROUGH THIN SKIN

(71) Applicant: Craig M. Gardner, Belmont, MA (US)

(72) Inventor: Craig M. Gardner, Belmont, MA (US)

(21) Appl. No.: 15/890,469

(22) Filed: Feb. 7, 2018

Related U.S. Application Data
(60) Provisional application No. 62/459,184, filed on Feb. 15, 2017.

(51) Int. Cl. A61B 5/1455

(52) U.S. Cl. CPC A61B 5/1455

(57) ABSTRACT

A transmission-mode optical spectroscopy method includes illuminating a first face of a skinfold with first light having plural wavelengths and detecting second light that emerges from a second face of the skinfold. The skinfold has a thickness of less than 1.5 millimeters.

### RELATED APPLICATIONS

Under 35 USC 119, this application claims the benefit of the Feb. 15, 2017 priority date of U.S. Provisional Application 62/459,184, the contents of which are herein incorporated by reference.

### FIELD OF INVENTION

This invention relates to non-invasive measurement of analytes in the blood, and in particular, to spectroscopy.

### BACKGROUND

Non-invasive measurement of analytes in blood is desirable because one can avoid having to be penetrated by a needle. A known way to carry out such analysis is to observe the interaction of blood and tissue with light. However, because tissue is filled with absorbing material, it tends to attenuate light passing through it. This is why, for example, human beings have a tendency to cast shadows.

### SUMMARY

In one aspect, the method includes carrying out transmission-mode optical spectroscopy by illuminating a first face of a skinfold with first light having plural wavelengths and detecting second light that emerges from a second face of the skinfold. The skinfold has a thickness that is below 1.5 millimeters.

Among the practices of the invention are those that include selecting the first and second wavelengths to be within a range that extends from 300-600 nanometers, those that include selecting the first and second wavelengths to be within a range that extends from 2000 and 2500 nanometers, those that include selecting the first and second wavelengths to be within a range that extends from 3400 and 5900 nanometers, and those that include selecting the first and second wavelengths to be within a range that extends from 7000 and 10,000 nanometers.

Other practices include determining where blood vessels are located in the skin fold and causing a distribution of the first light that promotes passage of the first light through the blood vessels or causing a distribution of the first light that promotes passage of the first light away from the blood vessels.

Yet other practices include those that include forming the skinfold and, after having done so, controlling a force applied to maintain the skinfold.

Other practices include heating at least one of the skinfold's two faces.

A variety of types of spectroscopy are contemplated to be within the scope of the invention, including Raman spectroscopy, fluorescence spectroscopy, and absorption spectroscopy.

The invention includes practices in which the skinfold is formed from a variety of anatomical features, including but not limited to skin on a penis, skin on an eyelid, skin in an inner labium, skin located in a region with no subcutaneous fat, skin located in a region with no hair follicles, skin found in a cherry angioma, and skin located in a raised mole.

Other practices include exploiting existing skinfolds that have the requisite thickness. Such skinfolds can be found in a skin tag or acrochordon.

In some practices, the first light comprises a range of wavelengths that includes first and second wavelengths. In some of these practices, the range is continuous. In others, the range is discrete.

Yet other practices include those that comprise measuring a thickness of the skinfold. Among these are embodiments that use a signal representative of the measured skinfold as a basis for feedback control, for example, for feedback control over force application, data processing, or data acquisition, or combinations thereof.

These and other features of the invention will be apparent from the following detailed description and the accompanying FIGURE, in which:

### BRIEF DESCRIPTION OF THE FIGURES

FIG. 1 is a block diagram of a system for transmitting light through skin.

### DETAILED DESCRIPTION

In many places on the body, it is possible to pinch the skin to form a skinfold. A skinfold extends in a direction essentially perpendicular to the plane of the surrounding skin.

An advantage of a skinfold is that it has first and second opposed faces. This means that it is possible to direct light through one face and received it through the other face. Since this light will necessarily pass through blood and tissue, the spectrum of the detected light will reveal the interaction of that blood and tissue with the incident light. From this, it is possible to infer what the constituents of the tissue might be.

As used herein, “light” refers to electromagnetic radiation that extends into the infrared range. Although such radiation has longer wavelengths than visible light, the technical problems that arise in manipulation of such radiation are not unlike those that arise in the visible range.

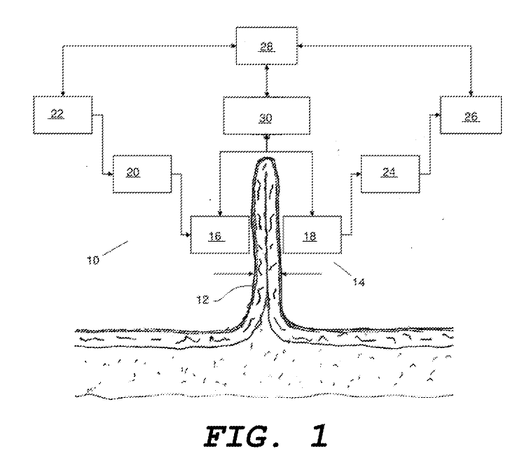

FIG. 1 shows a measurement apparatus 10 for measuring analyte levels by passing light through a skinfold 12. Examples of analytes within the blood include glucose, hemoglobin, total serum protein, albumin, globulin, immunoglobulin, fibrinogen, ethanol, sodium, chloride, potassium, bicarbonate, lactate, urea, creatinine, total cholesterol, HDL, LDL, triglyceride, chemotherapy drugs, and blood thinner drugs.

The apparatus features a clamp 14 having first and second faces 16, 18 for maintaining the skinfold 12. The first face 16 is coupled to a light director 20 that receives light from a light source 22. This light is thus transmitted into the skinfold 12 through the first face 16.

In some embodiments, the light source 22 is a broadband light source. However, in other embodiments, the light source 22 is a narrowband light source that is rapidly scanned across a range of wavelengths. In yet other embodiments, particularly those that rely on detecting fluorescence or on Raman spectroscopy, the light source 22 is a narrowband light source that is tuned to an excitation wavelength.

The second face 18, meanwhile couples to a light collector 24 that transmits light to a spectrometer 26. In some cases, the second face 18 includes a filter to attenuate selected frequencies of light transmitted through the skinfold. This can be useful for attenuating the excitation wavelengths so that mostly only the response wavelengths reach the detector. Embodiments include those in which the spectrometer 26 is configured to carry out absorption spectroscopy, fluorescence spectroscopy, or Raman spectroscopy.

The spectrometer 26 and the light source 22 are both coupled to a control system 28 having a suitable user interface.

In some embodiments, the clamp 14 connects to function unit 30 that carries out certain auxiliary functions that are useful for collecting data. In some embodiments, the function unit 30 includes an actuator that causes the clamp 14 to apply a variable clamping force to the skin.

In others, the function unit 30 features a heat source that applies heat to the clamp 14. In other embodiments, the function unit includes an imaging system. Among these embodiments are those in which the function unit 30 steers the incident light so that it is incident on different parts of the skinfold 12.

It is preferable that the skinfold 12 be between 0.3 mm and 1.5 mm thick and that the incident light have a wavelength between 300 and 600 nanometers, or that it have a wavelength between 2000 and 2500 nanometers, or that it have a wavelength between 3400 and 5900 nanometers, or that it have a wavelength between 7000 and 10,000 nanometers.

This combination of skinfold thickness and wavelength provides an element of criticality because it is such that a significant amount of light incident on the first face will escape absorption and exit the skinfold 12 through the second face. Yet, the tissue through which the light passes is thick enough so that there will be meaningful interaction between the tissue and blood and the light propagating through the skinfold 12.

A variety of sites on a typical body are amenable to such skin folds. These include the eyelid, which yields a skinfold 12 only 0.57 mm±0.10 mm thick, the inner labia, the inguinal region, a raised mole, or the penile shaft. The penile shaft is particularly suitable not only because the skin is only about 0.41 mm±0.03 millimeters thick but also because there is no subcutaneous fat and there are neither hair follicles nor sweat glands. As such, there are fewer structures and chemicals that could interfere with the accurate measurement of blood analytes. Additional locations that are suitable include skin tags, cherry angiomas, and acrochordons.

The skin of newborns and infants is known to be thinner than adults. Therefore, there may be additional sampling sites on newborns and infants that are in this thickness range.

When making quantitative measurements in transmission, it is important to know the actual thickness of the sample. This information is useful for a variety of purposes, among which are normalizing measured absorbance spectra. In some embodiments, the apparatus further includes a measurement scale to permit estimation of thickness. This measurement can be used as a feedback signal for feedback control over force adjustment, adjusting the data acquisition, and/or data processing steps.

A particular advantage of a thin skinfold 12 is that it becomes practical to determine where the blood vessels are actually located. To exploit this property, certain embodiments of the function element 30 include an imaging system configured to map the blood vessels that are in the field-of-view of the first face 16. The function element 30 in this embodiment also includes a mechanism for controlling which parts of the first face 16 are illuminated. This permits directing all of the light into blood vessels, thus avoiding wasting light by passing it through tissue that is unlikely to have any of the analytes of interest.

In some cases, one may wish to measure analyte in tissue and thus avoid directing light through blood vessels. This can be carried out in a similar manner.

In some embodiments, the imaging system of the function element 30 is able to carry out real-time tracking of the positions of blood vessels. This capability can be harnessed in either of the two applications described above to maintain optimal illumination through blood vessels.

In some embodiments, the function unit has a force transducer to measure the force exerted on the skinfold, and an actuator to apply a force. This is useful since it is often desirable to keep the applied pressure constant to avoid disturbing blood flow. Alternatively, one may wish to avoid having blood in the field-of-view, in which case it may be desirable to apply a larger force to exclude blood from the field-of-view. Yet another advantage of applying a higher force is the possibility of compressing the tissue, thereby creating the possibility of having more light be transmitted to the detector. This tends to increase the measurement's signal-to-noise ratio.

In some cases, it is useful to dilate blood vessels to promote increased blood flow during the measurement. For this purpose, the function element 30 includes a heat source. A suitable heat source is a resistive wire in the vicinity of the tissue, together with a power supply that causes current to flow through the resistive wire.

For many purposes, it is sufficient to obtain a single measurement. However, in some cases, it is desirable to monitor the concentration of an analyte as a function of time. In such cases, it is useful the controller can be configured to take measurements periodically, or essentially continuously. For extended use, certain embodiments of the apparatus are wearable.

Having described the invention, and a preferred embodiment thereof, what is claimed as new, and secured by Letters Patent is:

1. A method comprising carrying out transmission-mode optical spectroscopy, wherein carrying out transmission-mode optical spectroscopy comprises illuminating a first face of a skinfold with first light, said first light having at least first and second wavelengths, and detecting second light that emerges from a second face of said skinfold, wherein said skinfold has a thickness below a threshold thickness, wherein said threshold thickness is 1.5 millimeters.

2. The method of claim 1, further comprising determining where blood vessels are located in said skin fold and causing a distribution of said first light that promotes passage of said first light through said blood vessels.

3. The method of claim 1, further comprising determining where blood vessels are located in said skin fold and causing a distribution of said first light that promotes passage of said first light away from said blood vessels.

4. The method of claim 1, further comprising heating at least one of said first and second faces of said skinfold.

5. The method of claim 1, further comprising selecting said first and second wavelengths to be within a range that extends from 300-600 nanometers.

6. The method of claim 1, further comprising selecting said first and second wavelengths to be within a range that extends from 2000 and 2500 nanometers.

7. The method of claim 1, further comprising selecting said first and second wavelengths to be within a range that extends from 3400 and 5900 nanometers.

8. The method of claim 1, further comprising selecting said first and second wavelengths to be within a range that extends from 7000 and 10,000 nanometers.

9. The method of claim 1, wherein said first light comprises a range of wavelengths that includes said first and second wavelengths.

10. The method of claim 9, wherein said range is continuous.

11. The method of claim 9, wherein said range is discrete.

12. The method of claim 1, further comprising measuring a thickness of said skinfold.

13. The method of claim 12, further comprising using a signal representative of a measured skinfold as a basis for feedback control.

14. The method of claim 12, further comprising using a signal representative of a measured skinfold as a basis for feedback control over force application.

15. The method of claim 12, further comprising using a signal representative of a measured skinfold as a basis for feedback control over data acquisition.

16. The method of claim 12, further comprising using a signal representative of a measured skinfold as a basis for feedback control over data processing.

17. The method of claim 1, further comprising forming said skinfold and, after having formed said skinfold, controlling a force applied to maintain said skinfold.

18. The method of claim 1, further comprising forming said skinfold from skin on a penis.

19. The method of claim 1, further comprising forming said skinfold from skin on an eyelid.

20. The method of claim 1, further comprising forming said skinfold from skin located in a region with no subcutaneous fat.

21. The method of claim 1, further comprising selecting said skinfold to be formed by skin at a skin tag.
